(19) United States
(12) Patent Application Publication
(10) Pub. No.: US 2013/0052257 A1
(43) Pub. Date: Feb. 28, 2013

(54) ANTIMICROBIAL HYDROGEL WOUND DRESSING

(71) Applicants: AKRAM AHMAD AL MOUSA, RIYADH (SA); SALEM S. AL-DEYAB, RIYADH (SA)

(72) Inventors: AKRAM AHMAD AL MOUSA, RIYADH (SA); SALEM S. AL-DEYAB, RIYADH (SA)

(73) Assignee: KING SAUD UNIVERSITY, RIYADH (SA)

(21) Appl. No.: 13/218,399

(22) Filed: Aug. 25, 2011

(51) Int. Cl. A61L 15/42; A61P 17/02; B01J 19/12; A61K 31/7036

(52) U.S. Cl. 424/445; 514/39; 204/157.64

(57) ABSTRACT

The antimicrobial hydrogel wound dressing is a swellable polymer gel made from about 7-9% (wt/vol) polyvinyl alcohol (PVA), preferably 8.9%, about 0.1% (wt/vol) polyvinyl pyrrolidone (PVP), and about 1-2% (wt/vol) agar, preferably 1%, the balance (about 90%) being distilled water, the foregoing contents being crosslinked by gamma radiation at a dose of about 30 kGy. Prior to crosslinking by gamma radiation, an effective amount of a pair of antibiotics is added to the gel at room temperature. The antibiotics include about 10,000 IU of polymyxin B sulfate, and about 5 mg neomycin per gram of gel. The polymyxin provides effective protection against various forms of gram negative microorganisms, and the neomycin is a broad spectrum antibiotic that provides protection against various forms of gram positive microorganisms. The hydrogel has sufficient mechanical strength for use as a wound dressing, and is capable of absorbing water up to 900% of its volume.

### BACKGROUND OF THE INVENTION

1. Field of the Invention

The present invention relates to wound dressings, and particularly to an antimicrobial hydrogel wound dressing that incorporates a pair of antibiotics that, in combination, provide protection against both gram-positive and gram-negative microorganisms.

2. Description of the Related Art

Wound dressings have been used for centuries to promote healing, to protect damaged tissue from contamination by dirt and foreign substances, and to protect against infection. Recent studies have shown that a moist environment helps to promote healing of puncture wounds, abraded tissue, burns, and the like. This has led to renewed interest in hydrogel wound dressings, which are made from swellable polymers.

Hydrogel wound dressings provide several advantages over conventional wound dressings. Hydrogel polymers are hydrophilic, so that they absorb water, keeping the environment moist, thereby promoting healing, rehydrating dead tissues, and enhancing autolytic debridement. Hydrogels may be applied as a solid sheet or film having a backing with or without an adhesive border for use as either a primary or secondary dressing, or as an amorphous gel, usually requiring a secondary covering. Hydrogel dressings are often cool on the surface of the wound, helping to relieve pain. By absorbing water, hydrogels permit the transport of drugs through the network of crosslinked polymer.

However there are problems with hydrogel wound dressings. Some hydrogels have been found to lack sufficient mechanical strength, causing the wound dressing to shed, and sometimes to tear. Hydrogel wound dressings are unable to absorb much wound exudate, leading to proliferation of bacteria. Hydrogels made with chemical crosslinking agents will sometimes leave unreacted chemical crosslinking agent, which may be toxic, requiring costly purification or sterilization procedures. Hydrogels made with natural polysaccharides often experience degradation or deterioration of the polysaccharide over time, shortening the shelf life or useful life of the dressing and creating an environment conducive to the growth of microorganisms that may cause infection.

Consequently, hydrogel wound dressings require proper selection of components and their relative proportions and careful preparation procedures to ensure an effective, safe wound dressing. The proper combination of components, their relative proportions, and preparation procedures is often not predictable, but requires extensive experimentation. Thus, an antimicrobial hydrogel wound dressing solving the aforementioned problems is desired.

### SUMMARY OF THE INVENTION

The antimicrobial hydrogel wound dressing is a swellable polymer gel made from about 7-9% (wt/vol) polyvinyl alcohol (PVA), preferably 8.9%, about 0.1% (wt/vol) polyvinyl pyrrolidone (PVP), and about 1-2% (wt/vol) agar, preferably 1%, the balance (about 90%) being distilled water, the foregoing contents being crosslinked by gamma radiation at a dose of about 30 kGy. Prior to crosslinking by gamma radiation, an effective amount of a pair of antibiotics is added to the gel at room temperature. The antibiotics include about 10,000 IU of polymyxin B sulfate, and about 5 mg neomycin per gram of gel. The polymyxin provides effective protection against various forms of gram-negative microorganisms, and the neomycin is a broad-spectrum antibiotic that provides protection against various forms of gram-positive microorganisms. The hydrogel has sufficient mechanical strength for use as a wound dressing, and is capable of absorbing water up to 900% of its volume.

These and other features of the present invention will become readily apparent upon further review of the following specification and drawings.

### BRIEF DESCRIPTION OF THE DRAWINGS

FIG. 1 is a graph showing percentage gel content as a function of agar concentration in an antimicrobial hydrogel wound dressing according to the present invention.

FIG. 2 is a graph showing percentage gel swelling as a function of agar concentration in an antimicrobial hydrogel wound dressing according to the present invention.

FIG. 3 is a graph showing percentage gel content as a function of radiation dosage for a fixed agar concentration of 1% in an antimicrobial hydrogel wound dressing according to the present invention.

FIG. 4 is a graph showing percentage gel swelling as a function of radiation dosage for a fixed agar concentration of 1% in an antimicrobial hydrogel wound dressing according to the present invention.

FIG. 5 is a graph showing tensile strength as a function of agar concentration in an antimicrobial hydrogel wound dressing according to the present invention.

FIG. 6 is a graph showing force at break as a function of agar concentration in an antimicrobial hydrogel wound dressing according to the present invention.

FIG. 7 is a graph showing tensile strength as a function of radiation dosage at a fixed agar concentration of 1% in an antimicrobial hydrogel wound dressing according to the present invention.

FIG. 8 is a graph showing force at break as a function of radiation dosage at a fixed agar concentration of 1% in an antimicrobial hydrogel wound dressing according to the present invention.

Similar reference characters denote corresponding features consistently throughout the attached drawings.

### DETAILED DESCRIPTION OF THE PREFERRED EMBODIMENTS

The antimicrobial hydrogel wound dressing is a swellable polymer gel made from about 7-9% (wt/vol) polyvinyl alcohol (PVA), preferably 8.9%, about 0.1% (wt/vol) polyvinyl pyrrolidone (PVP), and about 1-2% (wt/vol) agar, preferably 1%, the balance (about 90%) being distilled water, the foregoing contents being crosslinked by gamma radiation at a dose of about 30 kGy. Prior to crosslinking by gamma radiation, an effective amount of a pair of antibiotics is added to the gel at room temperature. The antibiotics include about 10,000 IU of polymyxin B sulfate, and about 5 mg neomycin per gram of gel. The polymyxin provides effective protection against various forms of gram-negative microorganisms, and the neomycin is a broad-spectrum antibiotic that provides protection against various forms of grain-positive microorganisms. The hydrogel has sufficient mechanical strength for use as a wound dressing, and is capable of absorbing water up to 900% of its volume.

Polyvinyl alcohol (PVOH, PVA, or PVAI) is a water-soluble synthetic polymer. Polyvinyl alcohol is an odorless and tasteless, translucent, white or cream-colored granular powder. The structure of polyvinyl alcohol (compound I) is given below:

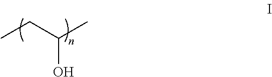

The physical characteristics and specific functional uses depend on the degree of polymerization and the degree of hydrolysis. Polyvinyl alcohol is classified into two classes, namely, partially hydrolyzed and fully hydrolyzed. Polyvinyl alcohol is prepared by hydrolyzing polyvinyl acetate (compound II) in alcohol in the presence of a base. Partially hydrolyzed PVA contains both PVA (compound I) and unreacted polyvinyl acetate or acetyl groups.

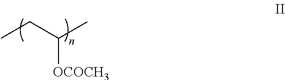

Commercially available PVA often has two numbers after the trade name, the first number indicating the degree of hydrolysis and the second number or numbers represents the viscosity. During the hydrolysis reaction, the acetate groups are hydrolyzed by ester interchange with the alcohol in the presence of a base. The physical characteristics and specific functional uses of PVA depend on the degree of polymerization and the degree of hydrolysis. Partially hydrolyzed PVA is used in the foods. Polyvinyl alcohol is a hydrophilic polymer and has excellent film forming, emulsifying, and adhesive properties. It is also resistant to oil, grease and solvent. It is odorless and nontoxic. It has high tensile strength and flexibility, as well as high oxygen and aroma barrier properties. However, these properties depend on humidity; in other words, with higher humidity, more water is absorbed. The water, which acts as a plasticizer, will then reduce tensile strength, but increase elongation and tear strength. PVA is fully degradable and is a quick dissolver. PVA has a melting point of 230° C. and 180-190° C. for the fully hydrolyzed and partially hydrolyzed grades, respectively. It decomposes rapidly above 200° C., as it can undergo pyrolysis at high temperatures.

Polyvinyl pyrrolidone (PVP) is a hydrophilic, water-soluble polymer having the structure shown in compound III:

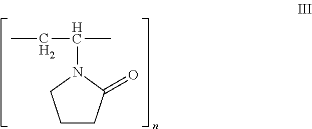

PVP is a nontoxic, swellable polymer that has also been used in hydrogel wound dressings. It may be crosslinked with PVA.

Agar is a polysaccharide complex extracted from algae. Agar has the following structure (compound IV):

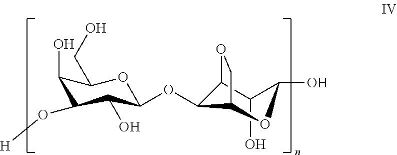

Agar is insoluble in cold water, but slowly soluble in hot water, forming a viscous solution. A 1% solution of agar forms a stiff jelly when cooled.

When crosslinked, PVA, PVP, and agar form a hydrogel that entraps water. The antimicrobial hydrogel wound dressing, prepared as described herein, contains about 90% water. In order to enhance the functionality and maintain the quality of the wound dressing, the hydrogel may serve as a vehicle for dispensing water-soluble topical antibiotics. However, antibiotics may interrupt the gelation process and may change the properties of the achieved gel in undesirable ways. After experimentation, the inventors have found that a combination of polymyxin B and neomycin may be incorporated into the wound dressing without impairing or adversely affecting polymerization or gel formation. Both polymyxin B and neomycin are known topical antibiotics, and they are often used together in topical applications. See Remington: The Science and Practice of Pharmacy, 21st ed., (2006), pp. 1651, 1654. The active ingredients of the well-known topical ointment Neosporin® (Neosporin is a registered trademark of Johnson & Johnson Corporation of New Brunswick, N.J.) are bacitracin, neomycin, and polymyxin B.

Polymyxin B is an antibiotic primarily used for resistant gram-negative infections. It is derived from the bacterium Bacillus polymyxa. Polymyxin B sulfate has a bactericidal action against almost all gram-negative bacilli, except the Proteus group. Polymyxins bind to the cell or cytoplasmic membrane and alter its structure, making it more permeable. The resulting water uptake leads to cell death. Polymyxin B is an N-monoacylated decapeptide in which seven of the ten amino acid residues are connected together in a ring configuration, having the structure shown in compound V below:

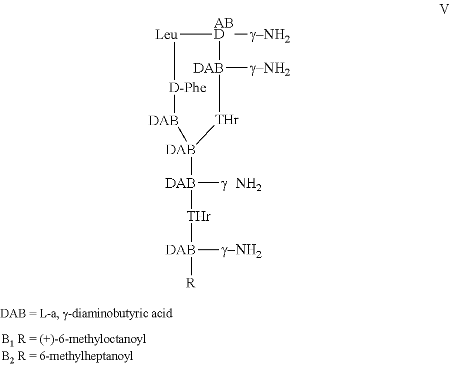

Polymyxin B may be separated into structures B1 and B2, which differ only in the length of the substituent on the side chain.

Neomycin is produced naturally by the bacterium Streptomyces fradiae. Neomycin is an aminoglycoside antibiotic that is found in many topical medications, such as creams, ointments, and eye drops. Similar to other aminoglycosides, neomycin has excellent activity against gram-negative bacteria, and has partial activity against Gram-positive bacteria (Staph and Enterococcus, but not streptococci). Neomycin has the structure of compound VI, shown below.

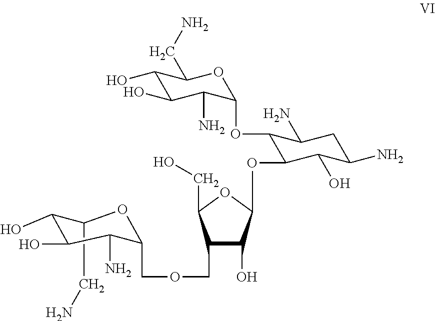

Optionally, the antimicrobial hydrogel wound dressing may include other excipients, such as preservatives (e.g., parabens, sorbates, and benzoates); humectants (e.g., polyethylene glycol [PEG]); and stabilizers.

Optionally, the antimicrobial hydrogel wound dressing may be attached to a backing material, which may be a cloth a fabric, a mesh, a foil, a foam, a net, and combinations thereof, which may be made from plastics, natural or synthetic fibers, paper, or metals.

The following example illustrates the antimicrobial hydrogel wound dressing described above.

### Example

The following materials were used. Polyvinyl alcohol (PVA) with average molecular weight 146,000-186,000 and degree of hydrolyzation of 99+% (compound I); polyvinyl pyrrolidone, (PVP), with an average molecular weight of 44,000, purchased from BDH Chemicals, England, (compound III); agar, purchased from DIFCO Laboratories, Detroit, Mich., USA, (compound IV); polymyxin B sulfate, purchased from MP Biomedical, USA, (compound V); and neomycin sulfate, purchased from Savniver Limited, China, (compound VI) were used for preparation of the wound dressing. All polymers, materials, and drugs were used without further purification.

For testing the antimicrobial properties of the wound dressing, cultures of the following microorganism were used in the study: gram-negative Escherichia coli, Pseudomonas aeruginosa, Klebsiella pneumonia, Proteus spp., and Salmonella spp. The pure cultures were obtained from the College of Science, Botany and Microbiology Dept., Research Central Laboratory, King Saud University, Saudi Arabia. Growth media was prepared from 5% peptone, 0.5% yeast extract, 1.5% agar and 0.5% NaCl per Liter of distilled water, and pH was adjusted to 7.0 at 25° C. Sodium phosphate buffer was prepared from 1 M of Na₂HPO₄ and 1 M NaH₂PO₄. This buffer was prepared as stock solution, and pH was adjusted to 7.0.

Double distilled water was used as solvent in all preparations.

The wound dressing was prepared as follows. 100 gm of PVA and 100 gm of PVP were heated in 1000 ml of distilled water for each polymer at 70-80° C. for 6 hours with stirring to have a 10% (wt/vol) PVA stock solution and a 10% (wt/vol) PVP stock solution. 99/1 parts of PVA/PVP were mixed together to have a total concentration of 8.9% (wt/vol) for PVA and 0.1% (wt/vol) for PVP. Different concentrations of agar (wt/vol) were used with the previous mixture of PVA/PVP (1%, 1.5% 1.75% and 2%). The agar amount was heated with the PVA/PVP mixture at 70-80° C. for 6 hours. The mixed solution was poured into a plastic mold or Petri dishes, covered with polystyrene sheet covers, and stored overnight at room temperature. The resulting gel was then irradiated by gamma rays with a series of doses (25, 30, 35, and 40 kGy) in order to crosslink the polymer and sterilize the wound dressing.

For those samples encapsulating one or more antibiotics, prior to irradiation, drug solutions with different concentration (see Table 1) were prepared by dissolving the drug in 2-3 ml of the corresponding solvent, and then the solution was mixed with the gel composition at room temperature and poured into the molds. Irradiation crosslinked the polymer and completed encapsulation of the antibiotic in the wound dressing.

TABLE 1
Concentration of antibiotics tested

| | Concentration | | | |
|---|---|---|---|---|
| Drug | IU | mg | Solvent | Remarks |
| Polymyxin B | 5000 | 0.6611 mg/1 gm of Gel | Water | |
| Polymyxin B | 10000 | 1.3222 mg/1 gm of Gel | Water | |
| Polymyxin B | 20000 | 2.644 mg/1 gm of Gel | Water | |
| Neomycin | — | 5 mg/1 gm of Gel | Water | 0.5% |

The first three samples listed in Table 1, using polymyxin B alone, were tested to check the inhibition of gram-negative bacteria. The results of the microbiological test, shown in Table 2, showed that the 5000 IU dosage was not enough to provide effective inhibition of the gram-negative bacteria. However, the samples with higher concentration of antibiotic (10,000 IU and 20,000 IU) were very promising.

TABLE 2
Tests of Polymyxin B only on gram-negative bacteria

| Concentration | Type of Bacteria | Inhibition (mm) |
|---|---|---|
| 5000 IU | All negative types | 0 |
| | Salmonella spp. | 3.5 |
| 10000 IU | Pseudomonas | 0 |
| | aeruginosa | |
| | Escherichia coli | 0 |
| | Salmonella spp. | 4.0 |
| 20000 IU | Pseudomonas | 0 |
| | aeruginosa | |
| | Escherichia coli | 3.0 |

The samples of wound dressings containing both polymnyxin B and neomycin were tested for antimicrobial activity for both gram-negative and grain-positive bacteria. The results of the microbiological test showed that the samples of 10,000 IU of polymyxin B and 5 mg of neomycin sulfate per each gram of gel was enough to make very good bacteria inhibition, as shown in Table 3.

TABLE 3
Tests of polymyxin B/neomycin combined

| Concentration | Strain of Bacteria | Inhibition (mm) | Type |
|---|---|---|---|
| 10000 IU | Pseudomonas | 10 | Gram Negative |
| Polymyxin B & | aeruginosa | | Bacteria |
| 5 mg Neomycin | Escherichia coli | 12 | |
| | Salmonella spp. | 14 | |
| | Escherichia coli | 3.0 | |
| | Staphylococcus | 12 | Gram Positive |
| | aureus | | Bacteria |
| | Streptococcus | 13 | |
| | pneumonia | | |
| | Bacillus cercus | 13 | |

Comparative testing was performed to optimize the percent gel content and the degree of swelling provided by the antimicrobial hydrogel wound dressing as a function of agar concentration and crosslinking radiation dosage, respectively.

During polymerization, crosslinking is not complete. In the final composition, besides the gel, a certain portion of PVA macromolecules is not joined by crosslinking, but forms a sol. The gel content, and therefore the degree of crosslinking of PVA and PVP, may be determined as follows. A sample of the hydrogel is dried until a constant weight is achieved, thereby indicating that all of the water has been removed. The mass of the dried hydrogel is measured and recorded. The dried hydrogel is then extracted with a suitable solvent, viz., water in order to remove any unreacted and uncrosslinked PVA and PVP, both of which are water soluble. The extracted hydrogel is again dried to a constant weight, and the mass is measured and recorded. The gel content G in the hydrogel is estimated as shown in Eq. 1:

$$G ( \% ) = \frac{{W}_{d}}{{W}_{1}} \times 100 \qquad ( 1 )$$

where W_{d} is the weight of the dried samples after extraction (only the crosslinked polymers) and W₁ is the weight of the dry sample before extraction, i.e., the combined weight of crosslinked and uncrosslinked polymers.

The degree of swelling could be described as the water absorptivity of the hydrogels. In order to determine the degree of swelling, the gel samples were immersed in distilled water with the proportion of the mass of the gel to the mass of water being about 1:500 at room temperature, the gel samples being for 48 hours. Swelling continued until the gel reached the equilibrium state of swelling, i.e., a constant weight of gel is achieved. After the water on the surface of the swollen gels was removed with cellulose paper, the mass was determined. The dried gels were obtained by drying at 50° C. until they reached a constant weight. The degree of swelling was defined as in Eq. 2:

$$Water absorptivity ( \% ) = \frac{{W}_{s} - {W}_{d}}{{W}_{d}} \times 100 \qquad ( 2 )$$

where: Wₛ is the weight of the swollen gels and W_{d} is the dried gel weight.

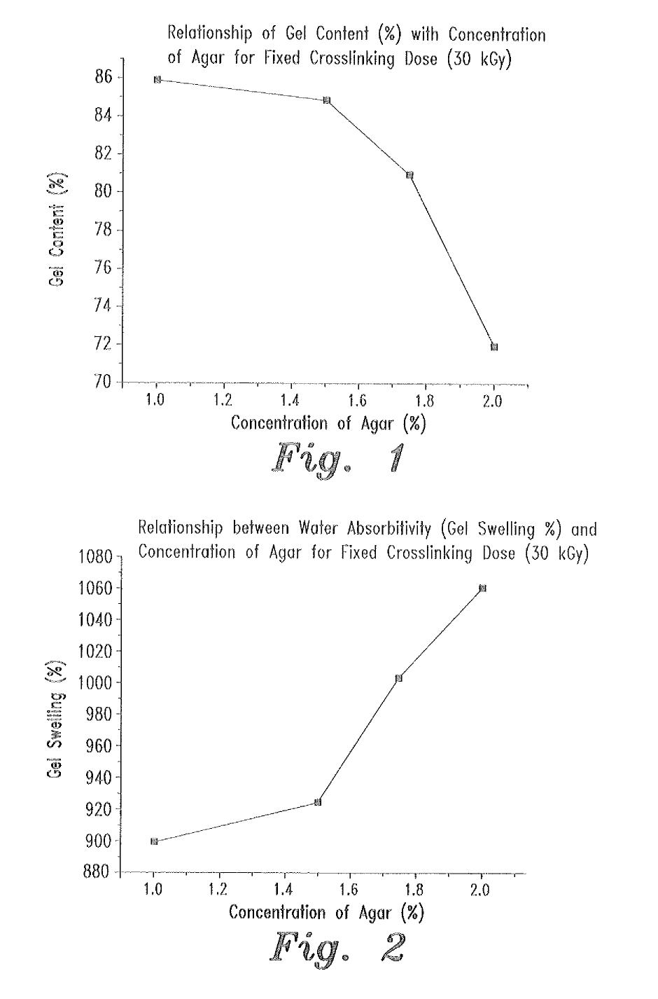

When crosslinking is kept constant by a gamma radiation dosage of 30 kilograys (kGy), then, as shown in Table 4 and FIG. 1, the gel content decreases with the increase of agar concentration. When the concentration of agar is from 1 to 1.5%, the gel content remains fairly stable and saturated; but further increase in the agar leads to a decrease in the gel content. On the other hand, water absorptivity is fairly stable between about 900-925% with an agar concentration between 1-1.5%, but increases almost linearly with increasing agar content above 1.5% (Table 4 and FIG. 2). The increase in agar concentration leads to higher swelling % or higher water absorptivity. A compromise has to be made to choose an agar concentration that has both good gel content % and good water absorptivity (Swelling %).

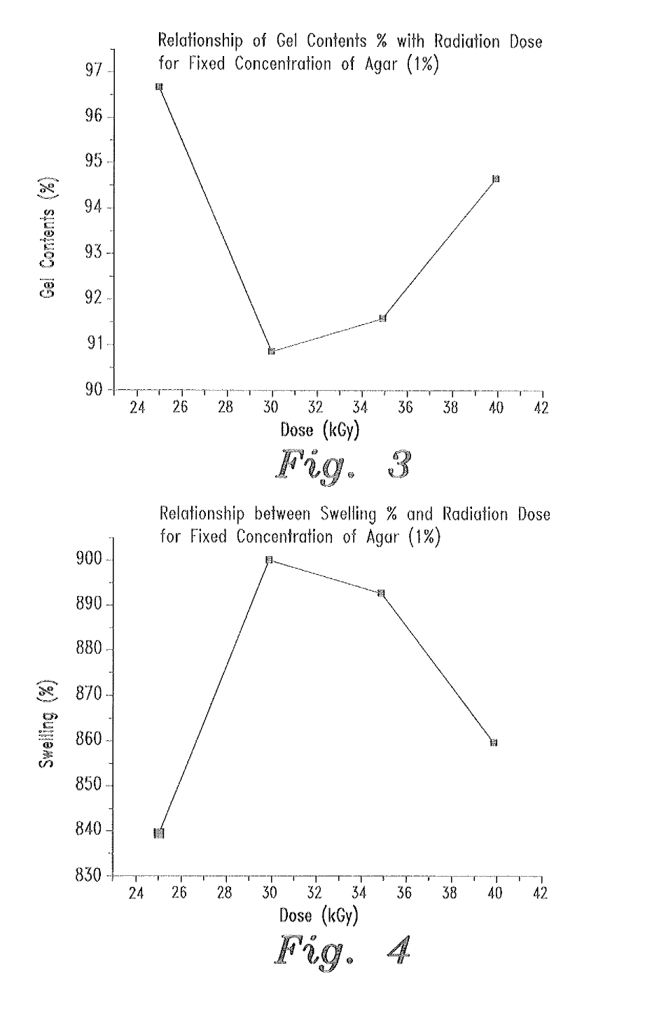

On the other hand, when agar concentration is kept constant at 1%, the effect of the dose change of gamma radiation in the range from 25 kGy to 40 kGy, as shown in Table 5 and FIGS. 3 and 4, was not big enough to make a difference. The gel content (%) and swelling (%) will not change significantly in this radiation dose range, which means that a change in the dosage of gamma radiation has a low or minimal effect on crosslinking and water absorptivity.

TABLE 4
Change of Gel Content % and Swelling % with Agar Concentration

| | Swelled | Dry Gel | Dry Gel | | | | |
|---|---|---|---|---|---|---|---|
| | Gel | After | Before | | | | |
| Agar | Weight | extract | extract | Gel | Av. Gel | Water | Av. Water |
| Concentration | (W0) | (Wd) | (W1) | Cont % | Cont % | absorptivity | Absor % |
| | 17.63 | 1.7 | 1.9393 | 87.66 | | 937.06 | |
| 1.00% | 17.6 | 1.8 | 1.936 | 92.98 | 85.87 | 877.78 | 899.98 |
| | 18.52 | 1.88 | 2.0372 | 92.28 | | 885.11 | |
| | 20.89 | 2.03 | 2.4024 | 84.50 | | 929.06 | |
| 1.50% | 21.5 | 2.08 | 2.4725 | 84.13 | 84.86 | 933.65 | 924.78 |
| | 20.03 | 1.98 | 2.3035 | 85.96 | | 911.62 | |
| | 25.55 | 2.3595 | 3 | 78.65 | | 982.86 | |
| 1.75% | 21.17 | 1.9066 | 2.49 | 76.57 | 80.98 | 1010.35 | 1003.41 |
| | 25.38 | 2.2721 | 2.59 | 87.73 | | 1017.03 | |
| | 17.44 | 1.55 | 2.0928 | 74.06 | | 1025.16 | |
| 2.00% | 16.04 | 1.29 | 1.9248 | 67.02 | 71.94 | 1143.41 | 1061.22 |

TABLE 5
Change of Gel content % and Swelling % with change of
radiation Dose for 1% Agar Concentration

| | | | | W. Dry | | | | |
|---|---|---|---|---|---|---|---|---|
| | | | W. Dry | Gel | | | | |
| | | W. of | gel after | before | Gel | Ave. Gel | | Ave. |
| Dose | | Gels (Ws) | ext (Wd) | ext (W1) | content % | Content % | Swelling % | Swelling % |
| 25 kGy | C(25)1 | 16.21 | 1.73 | 1.78 | 97.19 | 96.69 | 836.9942 | |
| | C(25)2 | 17.6 | 1.85 | 1.94 | 95.36 | | 851.3514 | 839.4273 |
| | C(25)3 | 14.6 | 1.57 | 1.61 | 97.52 | | 829.9363 | |
| 30 kGy | C(30)1 | 17.63 | 1.7 | 1.94 | 87.63 | 90.86 | 937.0588 | 899.9810 |
| | C(30)2 | 17.6 | 1.8 | 1.94 | 92.78 | | 877.7778 | |
| | C(30)3 | 18.52 | 1.88 | 2.04 | 92.16 | | 885.1064 | |
| 35 kGy | C(35)1 | 17.37 | 1.73 | 1.91 | 90.58 | 91.58 | 904.0462 | 892.5257 |
| | C(35)2 | 19.97 | 1.97 | 2.2 | 89.55 | | 913.7056 | |
| | C(35)3 | 21.98 | 2.29 | 2.42 | 94.63 | | 859.8253 | |
| 40 kGy | C(40)1 | 17.35 | 1.75 | 1.91 | 91.62 | 94.66 | 891.4286 | 859.5148 |
| | C(40)2 | 18.14 | 1.88 | 2 | 94.00 | | 864.8936 | |
| | C(40)3 | 16.6 | 1.8 | 1.83 | 98.36 | | 822.2222 | |

The effect of agar concentration and radiation dosage on the mechanical properties, particularly tensile strength and elongation at break, were also tested. The hydrogels were cut into a rectangular shape of 20 mm width and 3 mm thickness. The tensile strength and elongation at break of the PVA-PVP-agar blended hydrogel were measured using a Tinius Olsen-H5KS model universal testing instrument, with a load cell of −50 N, and with a crosshead speed of 50 mm/min.

The tensile test, also known as the tension test, is probably the most fundamental type of mechanical test one can perform on material. Tensile tests are simple, relatively inexpensive, and fully standardized. By pulling on something, the material behavior can be very quickly determined, and particularly how the material will react to forces being applied in tension. As the material is being pulled, its strength, as well as how much it will elongate, can be found. Many things can be learned about a substance from tensile testing. As one continues to pull on the material until it breaks, a good and complete tensile profile will be known. The curve that results shows how the material reacts to the forces being applied. The point of failure is of much interest, and is typically called its “Ultimate Strength” or UTS on the chart.

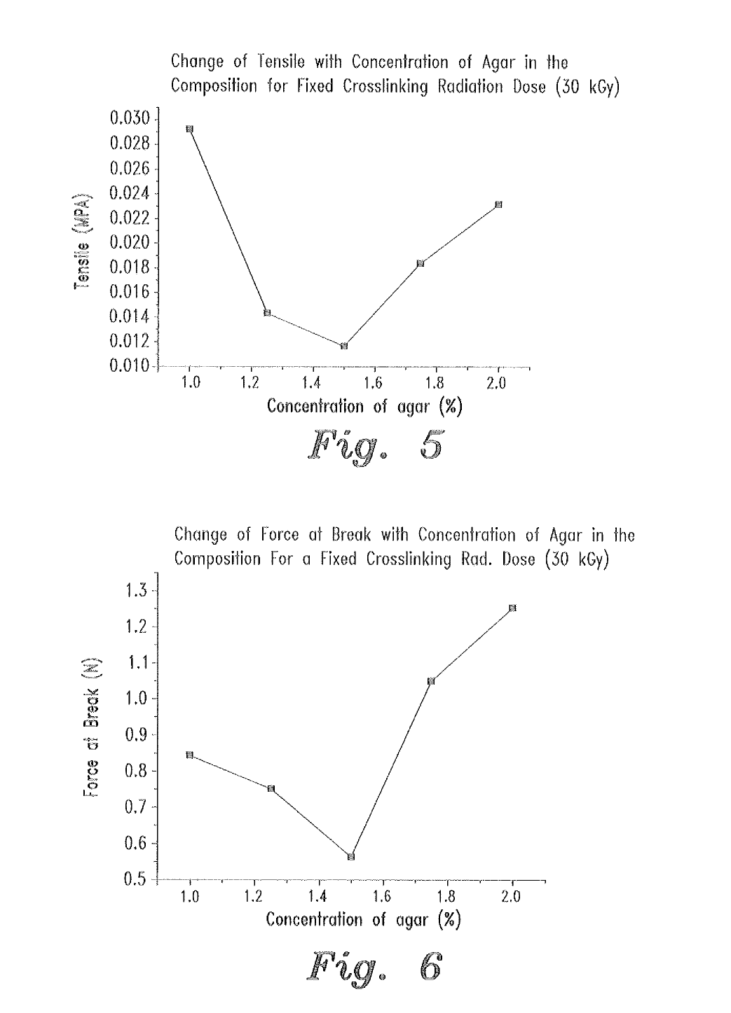

As shown in FIGS. 5 and 6, it is clear that tensile strength and force at break decreases until the concentration of agar is about 1.5%. After that, both tensile strength and force at break increase again. It is normal that the tensile strength decreases, due to the increase in the crosslinking in the composition with the increase in the polysaccharide concentration. However, the further increase in the agar concentration above 1.5% increases the portion of the polysaccharide (agar) that undergoes degradation by radiation, which increases the agar fragments in the composition that, in turn, will lead to a decrease in the crosslinking process and an increase in the tensile strength again.

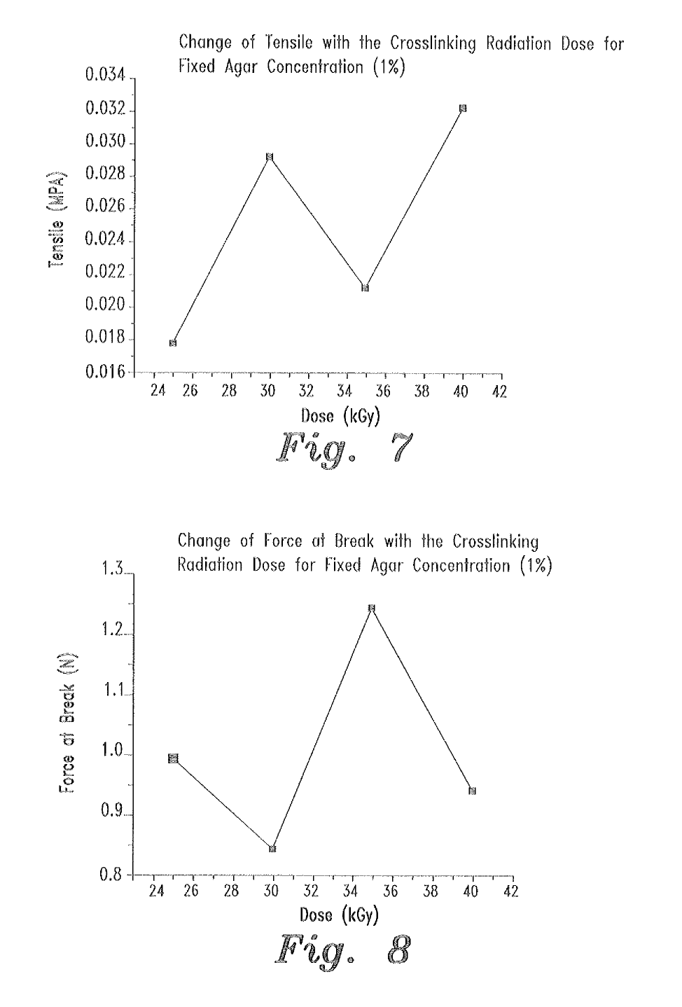

When the agar concentration is kept constant at 1% and the radiation dosage is varied between 25 kGy and 40 kGy, then, as shown in FIGS. 7 and 8, the force at break and tensile strength fluctuate around small values, which means close results. Using a concentration of agar from 1 to 2% and a crosslinking radiation dose from 25 to 40 kGy will give similar results. From the texture of the gels produced, we suggest that 1% concentration of agar and 30 kGy of radiation dose is sufficient to achieve the required crosslinking in the composition necessary to use the hydrogel as a wound dressing.

Chemically, when an aqueous solution of PVA (7-9%) containing polysaccharide, such as Agar (1-2%), is exposed to radiation, .OH, .H radicals, and hydrated electrons are produced, as major part of the radiation energy is absorbed by the solvent. .OH radicals are mostly responsible for crosslinking and degradation of PVA and polysaccharides, respectively. The rates of reaction of .OH radical with PVA and with polysaccharides, respectively, have similar bimolecular rate constants of the order of 10⁹ dm³ mol−¹ s−¹. Therefore, besides crosslinking of PVA, a fraction of the radicals would also degrade the polysaccharides in proportion to their concentration in aqueous PVA solution.

In conclusion, many trials for the preparation a new gel wound dressing were made using different compositions and using different types of polymers. Finally, a suitable gel made from about 8.9% (wt/vol) PVA/0.1% (wt/vol) PVP/1% (wt/vol) agar and about 90% water was achieved. The composition was crosslinked physically using around 30 kGy of gamma radiation. The physical crosslinking using gamma irradiation saved the use of chemical crosslinkers. Using this technique, both crosslinking and sterilization happen at the same time. A hydrogel with thickness of 2 to 4 mm that is easily handled and that can be used for wound dressing was prepared.

The wound dressing achieved was loaded with different topical antibiotics. Some of these antibiotics damaged the gel construction, and the crosslinking was interrupted due to free radical scavenging by the drug added. The wound dressing loaded successfully with two FDA-approved drugs for topical use. 10,000 IU polymyxin B and 5 milligram neomycin per each gram of gel were used. Microbiological assessment showed excellent inhibition for most gram-positive and gram-negative bacteria.

The new hydrogel wound dressing has a gelation percentage around 90% after crosslinking, and water swelling is about 900% of its weight.

Mechanical properties of the new gel with about 1% of Agar and with crosslinking at 30 kGy of gamma radiation showed 0.029 MPA tensile strength and 0.85 N force at break, which are enough so that the composition can be used as a wound dressing.

It is to be understood that the present invention is not limited to the embodiments described above, but encompasses any and all embodiments within the scope of the following claims.

We claim:

1. An antimicrobial hydrogel wound dressing, comprising:
a hydrophilic carrier having:
about 7-9% (wt/vol) polyvinyl alcohol (PVA);
about 0.1% (wt/vol) polyvinyl pyrrolidone (PVP);
about 1-2% (wt/vol) agar;
the balance (about 90%) being distilled water, the PVA and PVP being crosslinked by gamma radiation at a dose of about 30 kGy to form a hydrogel vehicle;
an effective amount of polymyxin B encapsulated in the carrier for inhibiting gram-negative microorganisms; and
an effective amount of neomycin encapsulated in the carrier for inhibiting gram-positive microorganisms.

2. The antimicrobial hydrogel wound dressing according to claim 1, wherein said PVA and said PVP are present in a ratio of about 8.9:0.1 prior to crosslinking.

3. The antimicrobial hydrogel wound dressing according to claim 1, wherein are between 80% and 90% crosslinked.

4. The antimicrobial hydrogel wound dressing according to claim 1, wherein said polymyxin B consists of between 5000 and 20000 IU of polymyxin B sulfate per gram of hydrogel.

5. The antimicrobial hydrogel wound dressing according to claim 1, wherein said polymyxin B consists of about 10000 IU of polymyxin B sulfate per gram of hydrogel.

6. The antimicrobial hydrogel wound dressing according to claim 1, wherein said neomycin consists of about 5 mg of neomycin sulfate per gram of hydrogel.

7. An antimicrobial hydrogel wound dressing, consisting essentially of:
a hydrophilic carrier having:
about 7-9% (wt/vol) polyvinyl alcohol (PVA);
about 0.1% (wt/vol) polyvinyl pyrrolidone (PVP);
about 1-2% (wt/vol) agar;
the balance (about 90%) being distilled water, the PVA and PVP being crosslinked by gamma radiation at a dose of about 30 kGy to form a hydrogel vehicle;
an effective amount of polymyxin B encapsulated in the carrier for inhibiting gram-negative microorganisms; and
an effective amount of neomycin encapsulated in the carrier for inhibiting gram-positive microorganisms.

8. The antimicrobial hydrogel wound dressing according to claim 7, wherein said PVA and said PVP are present in a ratio of about 8.9:0.1 prior to crosslinking.

9. The antimicrobial hydrogel wound dressing according to claim 7, wherein are between 80% and 90% crosslinked.

10. The antimicrobial hydrogel wound dressing according to claim 7, wherein said polymyxin B consists of between 5000 and 20000 IU of polymyxin B sulfate per gram of hydrogel.

11. The antimicrobial hydrogel wound dressing according to claim 7, wherein said polymyxin B consists of about 10000 IU of polymyxin B sulfate per gram of hydrogel.

12. The antimicrobial hydrogel wound dressing according to claim 7, wherein said neomycin consists of about 5 mg of neomycin sulfate per gram of hydrogel.

13. A method of forming an antimicrobial hydrogel wound dressing, comprising the steps of:
mixing polyvinyl alcohol (PVA), polyvinyl pyrrolidone (PVP), and agar with distilled water, the PVA and PVP being present in a ratio between about 70:1 and 90:1 by dry weight and the agar being present at about 1% (wt/vol) of the mixture;
heating the mixture to dissolve the agar to form a viscous solution;
cooling the viscous solution to form a stiff gel;
adding about 10000 IU of polymyxin B per gram of gel to the mixture;
adding about 5 mg of neomycin per gram of gel to the mixture; and
irradiating the gel with a dosage of about 30 kGy of gamma radiation to crosslink the PVA and PVP, the polymyxin b and the neomycin being encapsulated in the gel.

14. The method of forming an antimicrobial hydrogel wound dressing according to claim 13, wherein said step of heating the mixture comprises heating the mixture at 70-80° C. for 6 hours.

15. The method of forming an antimicrobial hydrogel wound dressing according to claim 14, wherein said step of cooling the mixture comprises the steps of;
covering the mixture; and
storing the covered mixture overnight at room temperature.
